(19) United States
(12) Patent Application Publication
(10) Pub. No.: US 2005/0113885 A1
(43) Pub. Date: May 26, 2005

(54) Patient notification of medical device telemetry session

(76) Inventors: Gregory J. Haubrich, Champlin, MN (US); Gregory J. Linden, Shorewood, MN (US); James E. Willenbring, St. Paul, MN (US); David S. Jennings, Minneapolis, MN (US); Charles L. Dennis, Lake Elmo, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/724,584

(22) Filed: Nov. 26, 2003

(51) Int. Cl. A61N 1/08

(52) U.S. Cl. 607/60; 607/32; 604/891.1

(57) ABSTRACT

Techniques are described for notifying a patient of initiation of a telemetry session with a medical device in order to monitor or reprogram parameters of the medical device. For example, a programming entity sends a notification to advise the patient of initiation of the programming session. The programming entity may send the notification to a notification device, which in turn conveys the notification to the patient. The patient performs an action in response to receiving the notification in order to indicate receipt of the notification to the programming entity. Upon receiving the notification from the patient, the programming entity initiates a programming session to reprogram one or more operating parameters of the medical device of the patient, or a telemetry session to interrogate the medical device.

### TECHNICAL FIELD

The invention relates to medical devices and, more particularly, to programming and interrogating medical devices by wireless telemetry.

### BACKGROUND

A programmable medical device is used to administer and deliver a therapy to a patient as well as monitor a condition of the patient. The programmable medical device may be an implantable medical device (IMD) that is implanted in the patient or an external medical device that is carried by the patient. One exemplary programmable medical device is an implantable cardiac device, such as an implantable pacemaker, an implantable cardioverter/defibrillator (ICD), or an implantable pacemaker/cardioverter/defibrillator (PCD). The implantable cardiac device, for example, delivers pacing pulses to a heart of the patient in order to produce a steady heart rhythm. Other exemplary programmable medical devices that administer and deliver therapies to the patient include neurostimulation devices, drug delivery devices, and the like.

The programmable medical device allows the patient to receive therapy without hospital admission and without direct assistance by a care provider. However, various operating parameters of the medical device may need to be reprogrammed from time to time due to variations in the patient's condition or responsiveness to therapy over time. In addition, the programmable medical device typically is interrogated to collect operational or physiological data stored in the medical device, or to monitor the current condition of the device or the patient. In each case, a telemetry session must be established with the medical device.

Generally, a telemetry session for a programmable medical device occurs at a clinic or in a hospital. In particular, the patient normally visits a clinic or hospital where the telemetry session is accomplished. In these circumstances, the patient is well aware that telemetry is taking place. In addition, the clinician or emergency personnel can ascertain the identity of the patient before initiating the telemetry session, and verify that the patient is in a physical location and position in which the telemetry session will be reliable and effective.

Telemetry sessions conducted at a clinic or hospital can result in difficulty and inconvenience, e.g., for patients living far away from the clinic or hospital or having limited physical mobility. Consequently, there has been considerable effort recently to provide medical devices with remote telemetry capabilities, e.g., for placement in the patient's home. Remote telemetry is typically conducted via a telephone line or wide area network connection. Under these circumstances, the patient may be unaware that a telemetry session is about to start. In addition, the patient may or may not be in a proper location or position to establish a reliable and effective telemetry session.

For example, in some cases, the patient may be positioned too far away from a remote telemetry station to ensure reliable telemetry. In other instances, operations incident to telemetry, such as reprogramming of parameters, may be undesirable due to the location or physical condition of the patient at the time reprogramming is initiated. For example, the patient may be ill or in the process of an activity such as work or exercise at the time of the reprogramming, and may not welcome a change in device parameters at that particular time.

Implantable medical devices have long had short range telemetry capabilities that permit programming and interrogation via a head with a radio frequency telemetry coil placed in proximity to the implanted device, e.g., within 3-5 cm. In addition to remote and short range telemetry, however, some implantable devices may provide local area telemetry, which permits longer range telemetry of greater than about 1-3 m.

With short range systems, the clinician or patient holds a transceiver directly over the implanted device. Given the very short range, it is clear with which implanted device the transceiver is communicating. With local area telemetry, however, it might be possible to have two or more patients and devices within range of the transceiver. For example, several patients within a clinic may be within a local area telemetry range. Accordingly, local area telemetry raises the possibility of initiating a telemetry session with an unintended device.

### SUMMARY

In general, the invention is directed to initiating telemetry sessions with a programmable medical device for programming, interrogation, or both. More particularly, techniques are described for notifying a patient of planned initiation of a telemetry session with a medical device carried by the patient, and, optionally, initiating the telemetry session in response to a patient action. The notification provides the patient with advance warning that a telemetry session is scheduled to take place, either imminently or in the near future. In response to the notification, the patient takes appropriate action, such as positioning himself for effective and reliable telemetry.

The optional feature of requiring patient action before the telemetry session can proceed ensures that the telemetry session will take place at an appropriate time in terms of patient comfort and safety. In addition, the patient action may provide an indication that the patient is aware of the telemetry session, and will position himself for effective and reliable telemetry. As a further feature, particularly for local area telemetry, the patient action may serve as a verification that the telemetry session has been initiated with the appropriate medical device, rather than a medical device carried by another patient.

In one embodiment, the invention provides a method comprising notifying a patient that has a programmable medical device of initiation of a telemetry session with the programmable medical device, and initiating the telemetry session in response to a patient action following the notification of the patient.

In another embodiment, the invention provides a system comprising a programmable medical device that performs at least one of diagnosing a patient, monitoring the patient, and delivering therapy to the patient and a notification device that notifies the patient of initiation of a telemetry session with the programmable medical device.

In a further embodiment, the invention provides a system comprising means for notifying a patient that has a programmable medical device of initiation of a telemetry session with the programmable medical device and means for initiating the telemetry session in response to a patient action following the notification of the patient.

In yet another embodiment, the invention provides a notification device comprising a communication unit that receives a notification of initiation of a telemetry session with a programmable medical device of a patient and an output medium to convey the notification to the patient to inform the patient of initiation of the telemetry session with the programmable medical device.

In another embodiment, the invention provides a method comprising receiving an electronic notification of initiation of a telemetry session with a medical device of a patient, conveying the electronic notification to the patient to inform the patient of initiation of the telemetry session with the programmable medical device and sending a response to the electronic notification upon receiving input from the patient.

In a further embodiment, the invention provides a method comprising sending an electronic notification to a patient that has a programmable medical device to notify the patient of initiation of a remote telemetry session with the programmable medical device.

In another embodiment, the invention provides a programming device comprising a notification generator that generates an electronic notification and a communication unit that sends the electronic notification to a patient that has a programmable medical device to notify the patient of initiation of a remote telemetry session with the programmable medical device.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a diagram illustrating a patient with a medical device participating in a telemetry session.

FIG. 2 is a block diagram illustrating a system for notifying a patient of planned initiation of a telemetry session with a medical device.

FIG. 3 is a block diagram illustrating the system of FIG. 2 in further detail.

FIG. 4 is a block diagram illustrating another exemplary system for electronically notifying a patient of planned initiation of a telemetry session with a medical device.

FIG. 5 is a schematic diagram illustrating another exemplary system for notifying a patient of planned initiation of a telemetry session with a medical device.

FIG. 6 is a block diagram illustrating a notification device for conveying a notification of a telemetry session to a patient in accordance with the invention.

FIG. 7 is a block diagram illustrating a programming device that notifies a patient of initiation of a telemetry session with a medical device carried by the patient.

FIG. 8 is a flow diagram illustrating notification of a patient concerning planned initiation of a telemetry session with a medical device carried by the patient.

FIG. 9 is a flow diagram illustrating exemplary operation of a notification system notifying a patient of initiation of a telemetry session with a medical device.

### DETAILED DESCRIPTION

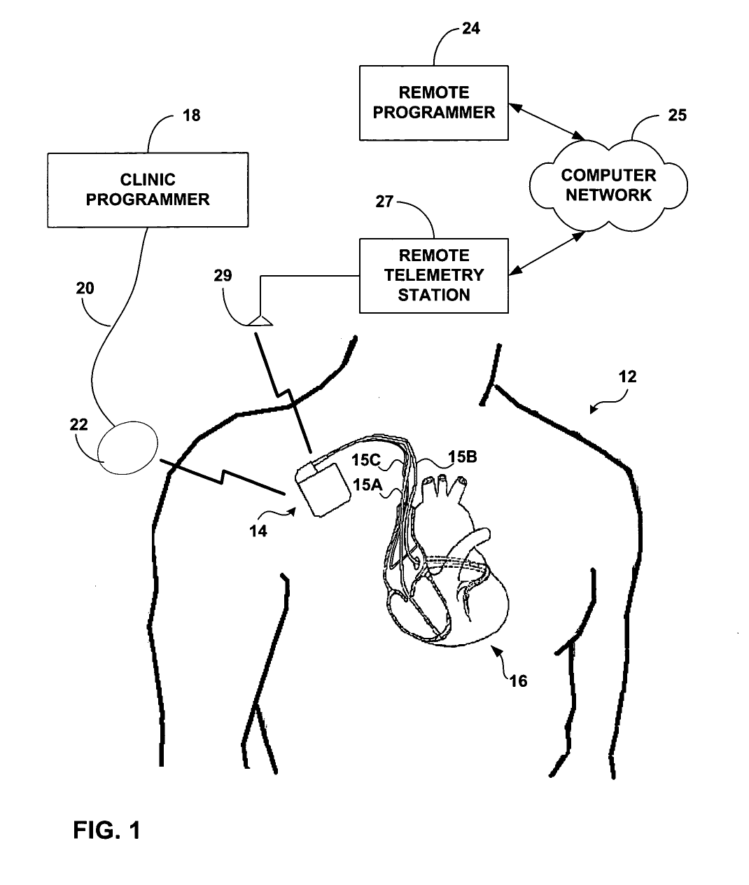

FIG. 1 is a diagram illustrating a patient 12 with a medical device 14 participating in a telemetry session. In accordance with the invention, techniques are described for notifying a patient of planned initiation of a telemetry session with a medical device carried by the patient and, optionally, initiating the telemetry session in response to a patient action. The notification provides the patient with advance warning that a telemetry session, with possible reprogramming of the medical device, is about to take place. In response to the notification, the patient may take appropriate action or preparation, such as positioning himself for effective and reliable telemetry, For example, patient 12 may position himself in close proximity to a remote telemetry station.

In the example of FIG. 1, medical device 14 is an implantable medical device (IMD), e.g., an implantable cardiac pacemaker-cardioverter-defibrillator, and will be referred to herein generally as an IMD 14. However, the invention may be applicable to other types of IMDs, such as implantable neurostimulators, drug delivery pumps, and the like. In addition, the invention is applicable to external medical devices carried by a patient 12. In either case, the medical device 14 is primarily therapeutic, primarily diagnostic, or combines both therapeutic and diagnostic and monitoring functions.

In the embodiment of FIG. 1, IMD 14 takes the form of a multi-chamber cardiac pacemaker. IMD 14 is implanted in a patient 12, and is coupled to right ventricular lead 15A, left ventricular coronary sinus lead 15B and right atrial lead 15C (collectively “leads 15”) that extend into the heart 16 of patient 12. Each of leads 15 includes electrodes (not shown), which IMD 14 uses to sense electrical signals attendant to the depolarization and repolarization of heart 16, and deliver pacing pulses or shocks. The configuration of IMD 14 and leads 15 illustrated in FIG. 1 is merely exemplary.

As further shown in FIG. 1, a clinic programmer 18 initiates a telemetry session with IMD 14 to interrogate or reprogram the IMD. Clinic programmer 18 resides within a clinic or hospital, and communicates with IMD 14 via a telemetry head 22, which is coupled to the clinic programmer via a cable 20. Telemetry head 22 is placed in proximity with IMD 14 to transmit and receive radio frequency (RF) signals. The telemetry session is a short range telemetry session via telemetry head 22. Hence, for telemetry with clinic programmer 18, patient 12 will visit a clinic or hospital.

A remote programmer 24 may be used to initiate a telemetry session with IMD 14 via a computer network 25 on a local area, wide area, or global network basis. Remote programmer 24 resides remotely from patient 12, who may be at home or at a remote satellite care facility. Alternatively, remote programmer 24 may be co-located within a clinical environment for local area telemetry with IMD 12, rather than short-range telemetry via a telemetry head.

For a patient at home, a remote telemetry station 27 communicates with remote programmer 24 via computer network 25, and facilitates communication with IMD 12 with a radio frequency antenna 29. In alternative embodiments, telemetry station 27 includes a telemetry head (not shown) similar to telemetry head 22. Accordingly, telemetry via remote programmer 24 may be conducted within a clinic on a local area basis, or within the patient's home using telemetry station 27 to support short range or local area network communication.

In accordance with the invention, programmer 18 or remote programmer 24 notifies patient 12 of initiation of the telemetry session before the telemetry session actually commences. The telemetry session with IMD 14 is an interrogation session, a reprogramming session, or a combination of both. The notification provides patient 12 with advance warning that a telemetry session, with possible reprogramming of IMD 14, is about to take place.

In response to the notification, in some embodiments, patient 12 takes appropriate action, such as positioning himself for effective and reliable telemetry. In addition, in some embodiments, patient 12 communicates a response to clinic programmer 18 or remote programmer 24. In each case, the notification and optional patient action can promote reliable telemetry and patient comfort and safety.

For example, clinic programmer 18 or remote programmer 24 can be configured to wait for an action from patent 12 before the telemetry session can proceed. In this manner, programmer 16 better ensures that the telemetry session will take place at an appropriate time in terms of patient comfort and safety. In particular, the patient action provides an indication that the patient is aware of the telemetry session, and will position himself for effective and reliable telemetry.

As a further feature, particularly for local area telemetry within a clinical setting, the action by patient 12 serves as a verification that the telemetry session has been initiated with the appropriate IMD 14, rather than an IMD carried by another patient. This feature is useful when a clinic or hospital is occupied by multiple patients with IMDs, and prevents the initiation of unauthorized or unintended telemetry sessions.

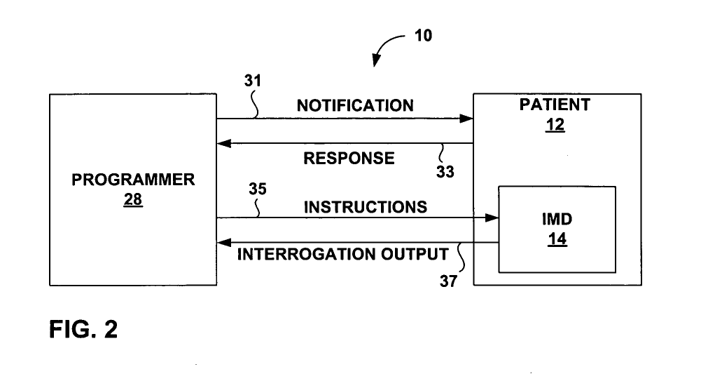

FIG. 2 is a block diagram illustrating a system 10 for notifying a patient 12 of initiation of a telemetry session with IMD 14 of patient 12. For purposes of illustration, the techniques of the invention will generally be described in terms of a programming session. However, it should be understood that the techniques are applicable to other types of telemetry sessions with IMD 14, such as an interrogation session, or sessions involving programming and interrogation.

In FIG. 2, a programmer 28 sends a notification to patient 12 to notify patient 12 of the initiation of a telemetry session, as indicated by line 31. Programmer 28 represents either clinic programmer 18 or remote programmer 24, both of FIG. 1. Remote programmer 24 operates in combination with telemetry station 27 (FIG. 1) if patient 12 is at home or otherwise away from a clinic or hospital. In either case, programmer 28 sends the notification to patient 12 via IMD 14 itself, or via a dedicated patient notification device, such as a telephone, a mobile phone, a pager, a personal digital assistant (PDA), a remote patient programmer or monitor, a computing device, e.g., a laptop computer, a desktop computer, or a workstation, a visual medium such as a web camera, postal mail, or the like.

Alternatively, a person, such as a messenger, could deliver the notification to patient 12. In each case, the notification includes, for example, patient identification information, such as a name, date of birth, Social Security number or the like, and telemetry session information indicating the need for the telemetry session as well as possibly a time, date or location at which the telemetry session shall occur.

In some embodiments, patient 12 performs an action in response to receiving the notification to indicate to programmer 28 that the notification was received. In this manner, the patient action acts as a response to the notification, and indicates to programmer 28 that patient 12 is aware that the telemetry session is needed, and that telemetry can proceed. In addition, the response from patient 12 indicates consent to initiation of the telemetry session.

In this manner, programmer 28 ensures that patient 12 is aware and approves of the initiation of the telemetry session. Ensuring the awareness and permission of patient 12 provides extra safety and security for patient 12 by ensuring that the operating parameters of IMD 14 will not be updated without the knowledge and potentially the permission of patient 12.

Notification and consent also avoids initiation of a telemetry session at a time at which changes in therapy or sensing parameters would be inconvenient or potentially dangerous to the patient. In addition, notification and consent confirms that the telemetry session is directed to the appropriate person. For example, in an environment in which several patients have medical devices capable of being programmed, notification and consent helps to ensure that a telemetry session has been targeted to the intended patient and medical device. Thus, notification and consent reduces the possibility of unauthorized or unintended programming or interrogation of a particular medical device.

Patient 12 sends a response to the notification to programmer 28, represented by line 33. The response to the notification takes a variety of forms. For example, in some embodiments, patient 12 actuates an input medium associated with a response device, which in turn sends the response to programmer 28. The input medium includes a wide variety of devices, such as buttons, keypads, keyboards, card readers, biometric readers, voice recognition devices, touch screens and the like.

In another example, the response device includes one or more sensors that detect a physical action performed by patient 12 in response to the notification, such as a posture or position change. In this case, the response device sends the response to programmer 28 upon detecting the physical action. If the patient is physically in the presence of the clinician or in other real-time interaction with the clinician, e.g., visual communication via a web camera, the patient simply tells the clinician orally. Otherwise, programmer 28 waits for communication of a response from patient 12 before proceeding with initiation of the telemetry session.

Programmer 28 initiates the telemetry session with IMD 14 upon receiving the response from patient 12. Specifically, programmer 28 communicates a set of instructions to IMD 14 to reprogram one or more operating parameters of IMD 14, as indicated by line 35. The set of instructions is communicated via one or more intermediate devices. For example, in some embodiments, a clinician interacts with a remote programmer 24 to relay the set of instructions via computer network 25 to remote telemetry station 27, which initiates the telemetry session with IMD 14.

In some embodiments, programmer 28 also receives interrogation output, indicated by line 37, from IMD 14. The interrogation output from IMD 14 includes, for example, operating parameters of IMD 14, which programmer 28 compares to the operating parameters contained within the set of instructions to verify that the desired operating parameters were properly updated during the telemetry session. The interrogation output also includes event data indicating sensed events, therapeutic events, and the like.

The invention may be applicable to any programmable medical device including an implantable medical device as well as an external medical device. For example, medical device 14 may be an implantable cardiac device such as a pacemaker, an implantable cardioverter/defibrillator (ICD), implantable pacemaker/cardioverter/defibrillator (PCD), or an implantable cardiac hemodynamic monitor, a subcutaneous (non-intravascular) monitor, a neurostimulation device, a drug delivery device, e.g., an insulin pump, or any other programmable medical device. Medical device 14 is used to diagnose patient 12, monitor patient 12, deliver therapy to patient 12, or a combination thereof. In some cases, medical device 14 may be a non-therapeutic device that primarily performs diagnosis or monitoring rather than therapy.

Again, the telemetry session established between programmer 28 and IMD 14 is either a remote telemetry session via remote programmer 24, computer network 25, and telemetry station 27, or an in-clinic telemetry session via clinic programmer 18. In general, the term “remote telemetry session” refers to a telemetry session that takes place without the physical presence of the clinician initiating the telemetry session. For example, the clinician is at a medical clinic while patient 12 is at home.

In another example, patient 12 is in a satellite medical clinic while the clinician initiating the telemetry session is in a primary medical clinic. As a further example, patient 12 is one of many patients within a clinic or hospital. In each case, remote telemetry involves initiation of a telemetry session outside of the direct presence of a clinician or other medical personnel responsible for programming, and is accomplished by a telecommunication link, either wired, wireless or a combination of both.

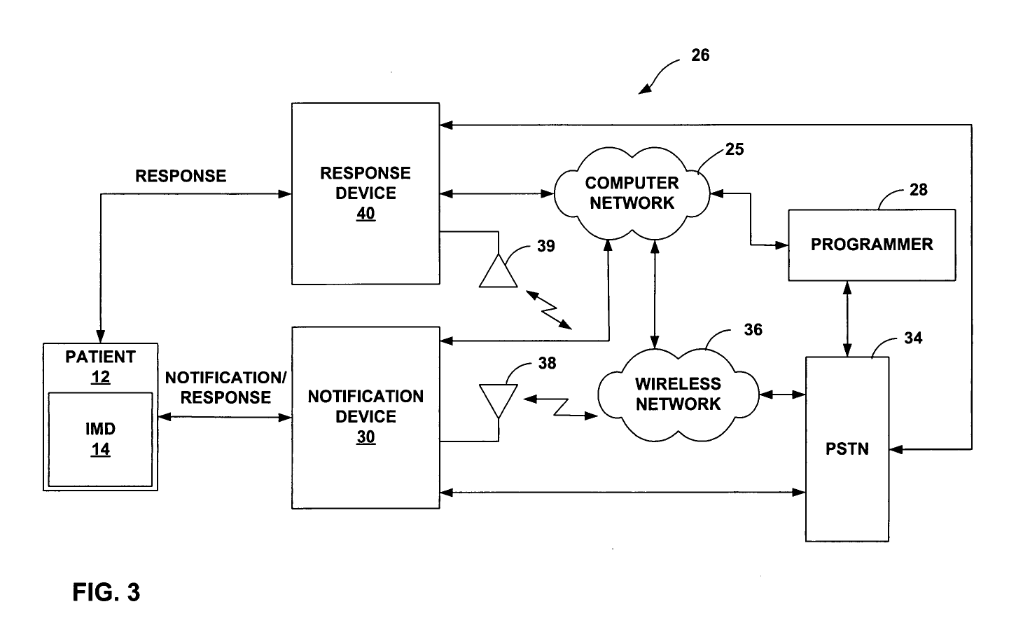

FIG. 3 is a block diagram illustrating an exemplary system 26 for notifying a patient 12 of initiation of a telemetry session with a medical device 14 in further detail. In accordance with one aspect of the invention, a programmer 28 generates and sends an electronic notification to patient 12 to notify the patient of initiation of a telemetry session to change one or more operating parameters of IMD 14. In some embodiments, programmer 28 makes use of a telephone, a mobile phone, a PDA, a clinician programmer, a computing device such as a laptop computer, a desktop computer, or a workstation, or other type of device capable of communicating with a notification device 30.

More specifically, programmer 28 sends the electronic notification to notification device 30, which conveys the notification to patient 12. Notification device 30, for example, conveys the electronic notification to patient 12 via a visible, audible or tactile medium. The notification medium is a visual display, such as a display screen or light emitting diode (LED), an audio speaker, a fragrance emitter, a vibrating element, an electrical stimulator or other output medium. In this manner, programming entity 16 notifies patient 12 of initiation of a telemetry session via a text notification, such as an electronic mail message, an instant message (IM) or a text message, an audible notification, such as a sound or a pre-recorded message, an odorous notification, or a tangible notification, such as vibration or a electrical stimulation.

Programmer 28, in some embodiments, automatically generates and sends the notification to patient 12 to advise patient 12 of initiation of a telemetry session. For example, in some embodiments, programmer 28 monitors performance of IMD 14 of patient 12 and automatically notifies patient 12 of initiation of a telemetry session with the IMD upon identifying the need to update operating parameters of the IMD or interrogate the IMD for current operating or patient status.

More typically, a telemetry session may be initiated by a member of a hospital or clinical staff. In particular, a programming operator, such as a clinician, interacts with programmer 28 to initiate the telemetry session. For example, the clinician analyzes information obtained from IMD 14 on a periodic basis to determine whether to update operating parameters of medical device 14. Also, in some embodiments, the telemetry session involves updating of software within IMD 14 without necessarily changing operating parameters.

In one embodiment, programmer 28 generates a notification and sends the notification to notification device 30 via computer network 25. For example, as described above, programmer 28 may generate a text notification, such as an electronic mail message, and send the electronic mail message to notification device 30 via computer network 25. In particular, routing devices (not shown), such as routers, switches, and the like, within computer network 25 relay the notification through the computer network to notification device 30.

Notification device 30 displays the electronic mail message to patient 12, thereby conveying the notification to the patient to inform the patient of initiation of the telemetry session. Computer network 25 may incorporate a combination of network architectures, including an Internet protocol (IP) network, a local area network (LAN), a wide area network (WAN), an asynchronous transfer mode (ATM) network, local wireless telemetry, or the like.

In yet another embodiment, programmer 28 communicates the notification to notification device 30 via a wireless network 36. For example, programmer 28 sends the notification through network 32 to a base station (not shown) within wireless network 36. Wireless network devices, such as base stations, within wireless network 36 generate signals that include the notification and send the notification signal to antenna 38 of notification device 30. Wireless network 36 and notification device 30 communicate using wireless techniques such as code division multiple access (CDMA), time division multiple access (TDMA), frequency division multiple access (FDMA) or some other modulation and multiple access techniques.

Although the above examples are described in terms of a text notification, the notification sent to patient 12 may be an audible notification, a tangible notification, an odorous notification or other type of notification method supported by notification device 30, as described above. In some embodiments, for example, programmer 28 communicates the notification to notification device 30 via a public switched telephone network (PSTN) 34. In particular, programmer 28 can be configured to automatically initiate a call to notification device 30 and play a pre-recorded message informing patient 12 of the need to initiate the telemetry session.

Notification device 30 comprises a telephone, a mobile phone, a pager, a PDA, a remote patient programmer, a remote monitor, a handheld programmer, a computing device such as a laptop computer, a desktop computer, or a workstation, a web camera, or other type of device capable of communicating with a programmer 28.

In some embodiments, patient 12 performs an action in response to receiving the notification to indicate receipt of the notification. In particular, the action performed by patient 12 causes a response to be sent to programmer 28 to indicate receipt of the notification. Additionally, patient 12 can be required to perform a series of actions to generate a response to the notification. The response to the notification assures a person operating programmer 28 that patient 12 is aware of and approves of the initiation of the telemetry session to update one or more operating parameters of IMD 14.

In some embodiments, patient 12 interacts with notification device 30 to respond to the received notification. In this manner, notification device 30 integrates both notification and response functionality. For example, patient 12 interacts with an input medium of notification device 30 to send a response to the notification to programmer 28.

In one example, patient 12 pushes a button of notification device 30, which in turn generates and sends a response to the notification. In another example, patient 12 interacts with a biometric scanner, such as a fingerprint scanner or a retinal scanner, to identify patient 12 and send the response upon positively identifying patient 12. Other interactions with the input medium of notification device 30 include, for example, entering a password, swiping a identification card through a card reader, speaking into a voice recognition device or the like.

Alternatively, patient 12 interacts with a dedicated response device 40, independent of notification device 30, to generate a response to the notification. Patient 12 interacts with response device 40 in the same manner as described above with respect to notification device 30. Response device 40 generates a response due to the interaction with patient 12 and sends the response to programmer 28 via any of wired network 25, PSTN 34, or wireless network 36 via antenna 39. In this manner, patient 12 can receive the notification from a first communication device, i.e., notification device 30, and respond to the notification via a different communication device, i.e., response device 40.

In one example, patient 12 receives the notification from a pager and sends a response to the notification by entering a code into a remote telemetry station 26 (FIG. 1) to initiate the telemetry session. Alternatively, response device 40 may comprise a telephone, a mobile phone, a PDA, a remote telemetry station, a remote monitor, a handheld programmer, a web camera, a computing device such as a laptop computer, a desktop computer, or a workstation, or other type of device capable of communicating with a communication device 28. In another example, patient 12 personally responds to the notification, e.g., via a visit to a clinic, an oral response or sending a response via postal mail. In some embodiments, the programming operator is unable to initiate the telemetry session until a response from patient 12 has been received.

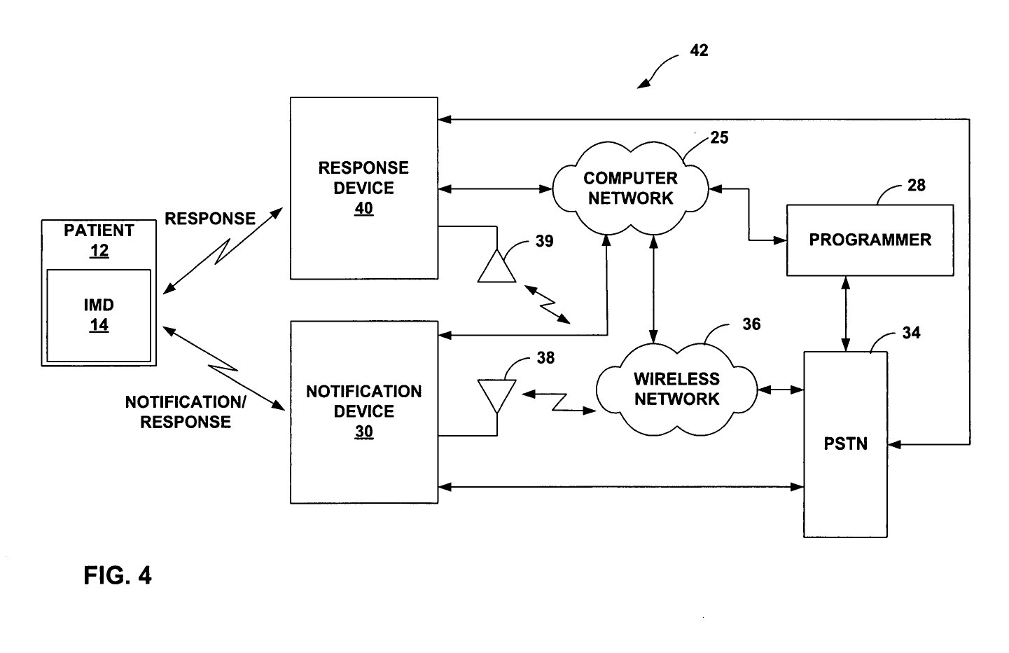

FIG. 4 is a block diagram illustrating another exemplary system 42 for notifying a patient 12 of initiation of a telemetry session, e.g., to update one or more operating parameters of IMD 14 of patient 12, or interrogate the IMD. System 42 conforms substantially to system 26 of FIG. 2, but notification device 30 and response device 40 do not directly interact with patient 12. Instead, notification device 30 and response device 40 communicate directly with IMD 14, which in turn notifies patient 12 of initiation of the telemetry session.

Programmer 28 generates and sends a notification to notification device 30 via computer network 25, PSTN 34, or wireless network 36. Notification device 30 transmits a signal to IMD 14 using wireless telemetry techniques. IMD 14 notifies patient 12 of initiation of the telemetry session upon receiving the signal from notification device 30.

IMD 14 can notify patient 12 of the need for the telemetry session using an audible notification, such as emitting a sound, or a tactile stimulation notification, e.g., vibrating IMD 14, stimulating a tissue site adjacent IMD 14, shocking patient 12, or the like. Particularly in the case of an external medical device, the medical device may flash a light, provide text notification, provide olfactory notification or use any of the other notification mechanisms described above for notification device 30. In reply to the notification, as in the example of FIG. 3, patient 12 performs an action to indicate to a programming operator receipt of the notification. The action may be carried out using an external response device 40.

Alternatively, IMD 14 may function as a response device. As one example, patient 12 swipes a magnet over IMD 14 in response to the notification. IMD 14 detects the magnetic field caused by the magnet and sends a response to programmer 28 to indicate receipt of the notification upon detecting the magnetic filed, thereby ensuring that patient 12 is aware of and approves of the need for initiation of the telemetry session.

As further alternatives, medical device 14 may be configured to detect other patient actions such as patient 12 holding his or her breath or otherwise modifying his or her breathing pattern for a specified period of time, breathing in a specified pattern, which is detected by respiratory sensors, tapping medical device 14 or an area of the body, e.g., skin, near an implantable medical device, which is detected by an accelerometer or other sensor, changing position of a body of patient 12 in a defined sequence (e.g., bending down and then standing up), which is detected by an accelerometer, shining a light at skin covering medical device 14, which is detected by an optical sensor, holding an antenna over the medical device, turning on a remote programming or monitoring device, or the like.

In some embodiments, clinic or hospital staff notify patient 12 of initiation of a telemetry session with IMD 14 by a personal notification. In one example, the hospital or clinic staff personally notifies patient 12 by sending a person to make a home visit to patient 12. For example, a clinician, a nurse or other medical personnel travel to a home of patient 12 to notify patient 12 of initiation of a telemetry session to update one or more operating parameters of IMD 14. In this example, patient 12 responds to the notification verbally, signs a declaration stating that patient 12 has received the notification, or sends the response via a notification or response device as described above.

In another example, a personal notification may be sent to patient 12 via postal mail 54. For example, clinical or hospital staff sends patient 12 a letter that informs patient 12 of initiation of the telemetry session at a particular date and time. Patient 12 sends, for example, a reply via postal mail 68 in response to the notification. Alternatively, patient 12 sends the response via the notification or response device as described in detail above.

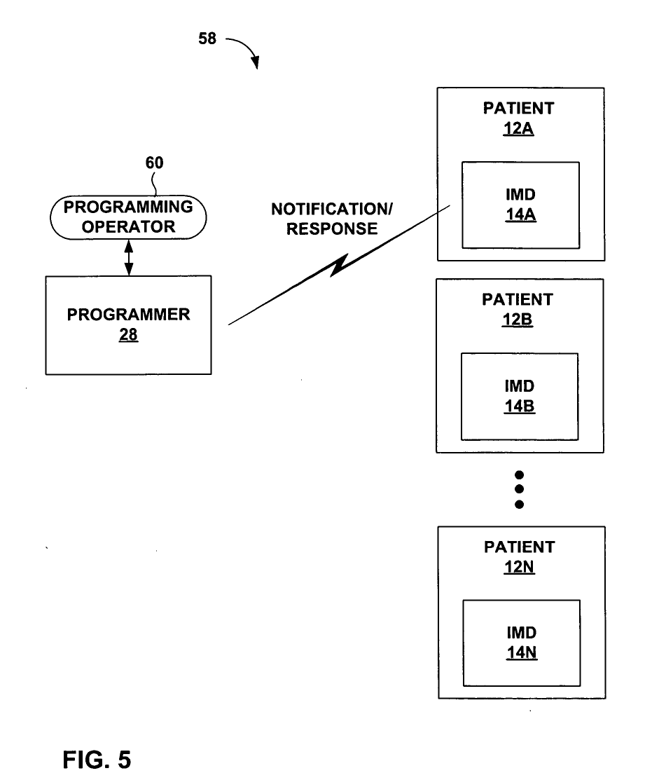

FIG. 5 is a schematic diagram illustrating another exemplary system 58 for notifying patient 12 of initiation of a telemetry session with a IMD 14. In the example illustrated in FIG. 5, programming operator 60 interacts with a programmer 28 to send an electronic notification to one of a plurality medical devices 14A-14N (“medical devices 14”) of a respective one of patients 12A-12N (“patients 12”) to ensure programmer 28 is in communication with an appropriate IMD 14.

As programmers become able to wirelessly communicate with IMDs at larger separation distances, the need to verify communication with the appropriate IMD 14 is essential. In FIG. 5, the plurality of patients 12 is located in a clinic, e.g., in adjacent rooms at the clinic, and programmer 28 potentially communicates with any of IMDs 14 of patients 12 due to the extended range of programmer 28 and the proximity of the patients. By sending an electronic notification to the appropriate IMD 14, in turn notifying a respective patient 12, and receiving a response from the patient, programming operator 60 ensures that telemetry has been established with the appropriate one of IMDs 14.

As an example, programmer 28 sends an electronic notification to one of the IMDs 14 that notifies a respective patient 12 of initiation of the telemetry session. The receiving medical device 14 emits a sound, stimulates a tissue site adjacent IMD 14, vibrates, or the like to provide the notification to the patient.

In response, patient 12 performs an action, such as verbally notifying programming operator 60 that the notification, e.g., the stimulation of the tissue site, was received and that the telemetry session can be initiated. Upon initiating the telemetry session with the appropriate one of IMDs 14, programmer 28 sends a set of instructions to respective IMD 14 in order to update one or more operating parameters of the IMD, or interrogates the IMD.

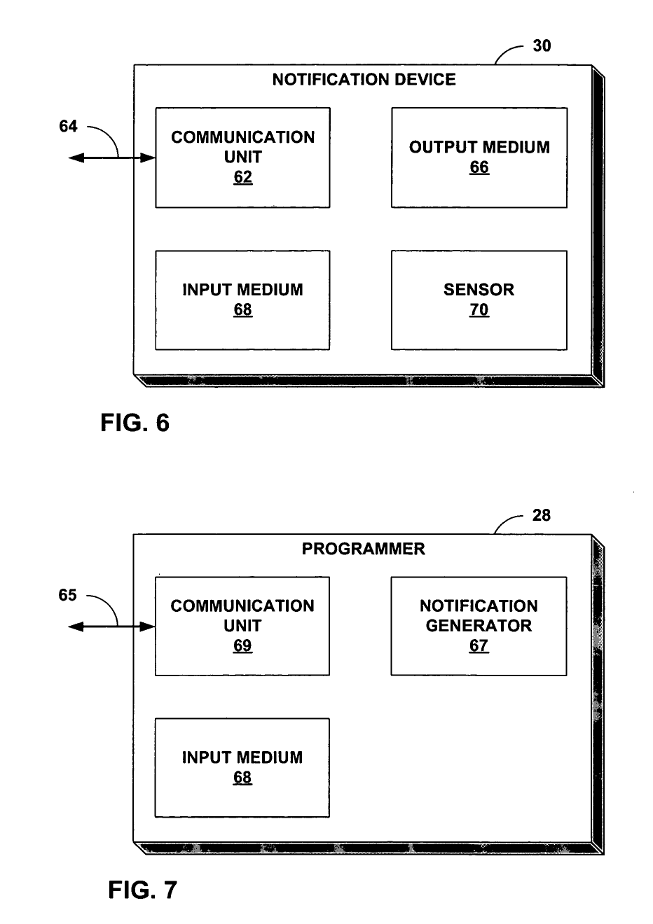

FIG. 6 is a block diagram illustrating a notification device 30 in accordance with the invention. As described above, notification device 30 receives a notification from programmer 28 and conveys the notification to patient 12 to advise the patient of initiation of a telemetry session with IMD 14. In some embodiments, directed to in-clinic or in-hospital programming, notification device 30 may be integrated with programmer 28.

Notification device 30 includes a communication unit 62 that is communicatively coupled to programmer 28 (FIG. 1). Communication unit 62 is coupled to computer network 25, PSTN 34, or wireless network 36 via at least one communication link 64. Communication unit 62 includes a network card, a wireless network card, an infrared (IR) network card, a modem, an antenna or any combination thereof. Communication link 64 is either a wired communication link or a wireless communication link. Communication unit 62 receives the notification from programmer 28 via communication link 64.

Notification device 30 conveys the notification to patient 12 via an output medium 66. Output medium 66 includes, for example, a display, such as a Liquid Crystal Display (LCD), a plasma display or a Light Emitting Diode (LED) display, a speaker, a fragrance emitter, a stimulator, or the like.

Notification device 30 can also include an input medium 68 by which patient 12 interacts with notification device 30. Input medium 68 is a keyboard, a keypad, a touch screen display, or a peripheral pointing device, such as a stylus or mouse, a card reader, a biometric reader, such as fingerprint scanner or retinal scanner, or a voice recognition device.

Patient 12 interacts with notification device 30 using input medium 68 in order to respond to the notification of the telemetry session. Notification device 30 includes, in some embodiments, at least one sensor 70 that detects a patient action in response to the notification. Sensor 70 includes, for example, a blood oxygenation sensor, an accelerometer, an optical sensor, or the like, which detects patient action and generates a signal indicative of the action.

Notification device 30 generates and sends a response to the notification upon sensor 70 detecting the patient action. For example, notification device 30 generates and sends a notification upon a blood oxygenation sensor reaching a particular level, thus indicating that patient 12 is holding his or her breath for a specified period of time.

FIG. 7 is a block diagram illustrating a programmer 28 that programs an IMD 14 in accordance with the invention. As described above, programmer 28 generates an electronic notification and sends the electronic notification to patient 12 to advise the patient of initiation of a telemetry session with medical device 14.

Programmer 28 includes a notification generator 67 that generates an electronic notification to send to patient 12 via notification device 30. The electronic notification includes, for example, patient identification information, such as a name, date of birth, Social Security number or the like, and telemetry session information indicating the need for the telemetry session as well as possibly a time, date or location at which the telemetry session shall occur.

Programmer 28 either automatically generates the electronic notification or generates the notification in response to receiving input from a programming operator 60 via input medium 68. For example, programmer 28 monitors performance of IMD 14 of patient 12 and automatically notifies patient 12 of initiation of a telemetry session with the IMD upon identifying the need to update operating parameters of the IMD. Again, in some embodiments, a notification device 30 may be integrated with programmer 28.

In addition, programmer 28 includes a communication unit 69 that is communicatively coupled to notification device 30 (FIG. 1). Communication unit 69 is coupled to computer network 25, PSTN 34, or wireless network 36 via at least one communication link 65. Communication unit 69 includes a network card, a wireless network card, an infrared (IR) network card, a modem, an antenna or any combination thereof. Communication link 65 is either a wired communication link or a wireless communication link. Communication unit 69 sends the electronic notification to notification device 30 via communication link 64.

Programmer 28 receives a response from patient 12 and initiates a telemetry session with medical device 14 of patient 12 upon receiving the response. In this manner, programmer 28 assures that patient 12 is aware of the telemetry session. Programmer 28 either directly communicates with IMD 14, e.g., during an in-clinic telemetry session, or communicates with the IMD through one or more intermediate devices, e.g., during a remote telemetry session. In one example, communication unit 69 includes wireless telemetry circuitry and an antenna to wirelessly communicate with IMD.

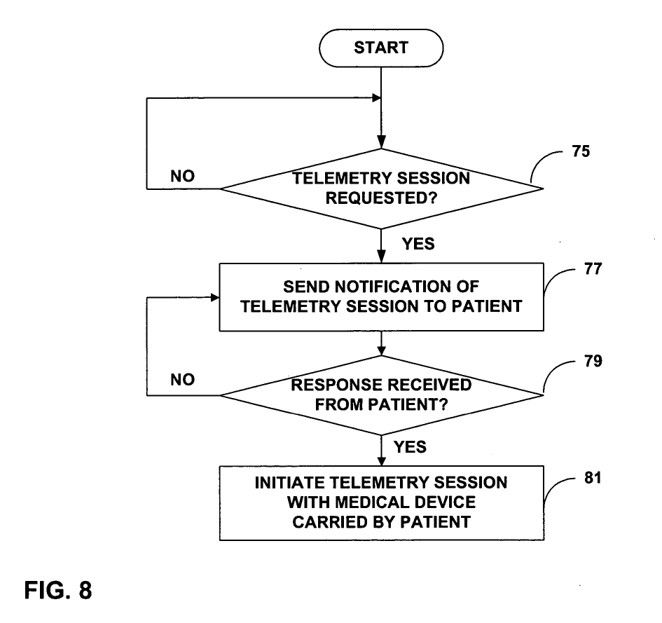

FIG. 8 is a flow diagram illustrating notification of a patient concerning planned initiation of a telemetry session with an IMD 14 carried by the patient 12. As shown in FIG. 8, when a telemetry session is requested by a programmer 28 (75), the programmer sends a notification of the telemetry session to the intended patient 12 (77) via any of the techniques described herein. Optionally, the programmer 28 may await a response from the patient 12 (79). Upon receipt of the response, the programmer 28 initiates the telemetry session with the IMD 14 carried by the patient (81).

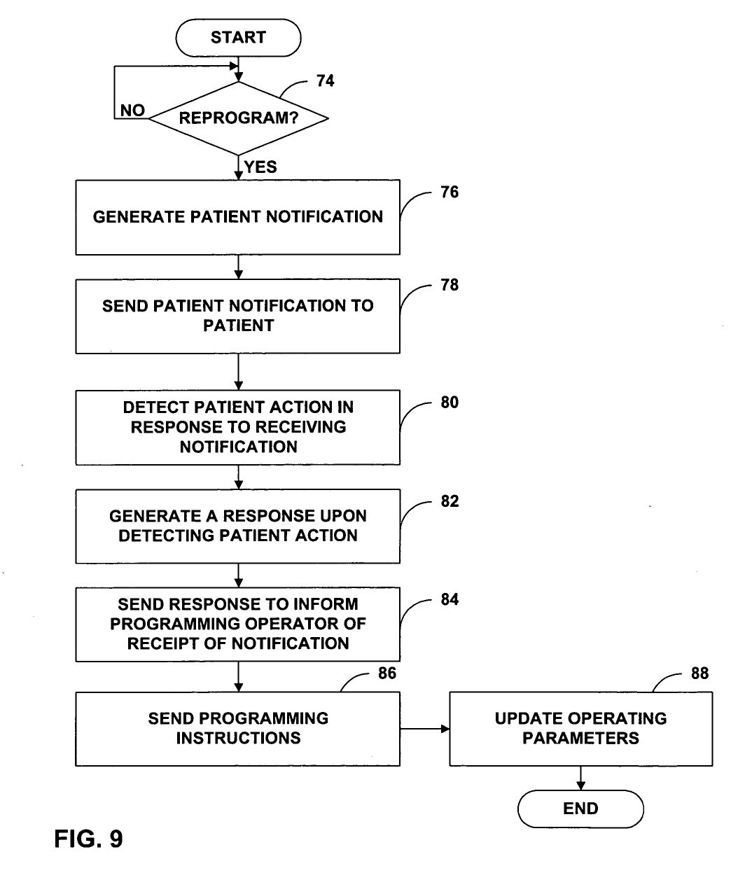

FIG. 9 is a flow diagram illustrating exemplary operation of a notification system for notifying a patient 12 of initiation of a telemetry session with IMD 14. Initially, the notification system determines whether IMD 14 of patient 12 needs to be reprogrammed with updated operating parameters (74), or whether the IMD should be interrogated to obtain operational or patient data. In some embodiments, a clinician or other medical personnel monitors parameters obtained by IMD 14 to determine whether the operating parameters of the IMD are effectively treating patient 12.

If the operating parameters do not result in effective treatment of patient 12, the notification system notifies the patient of initiation of the telemetry session with IMD 14. Specifically, a programmer generates a notification (76) and sends the notification to notification device 30 (78). The programmer automatically generates the notification or interacts with a clinician to generate the notification. The programmer sends the notification to notification device 30 via computer network 25, PSTN 34, or wireless network 36 as described in detail above.

Notification device 30 conveys the notification to patient 12. Notification device 30, for example, conveys the notification to patient 12 via an output medium such as a display screen, LED, speaker, or the like. In another embodiment, notification device 30 communicates the notification to IMD 14 via wireless telemetry techniques and medical device 14 conveys the notification to patient 12 via electrical stimulation of a tissue site, vibration of medical device 14 or emitting a sound. Alternatively, patient 12 receives the notification in-person from a home visit nurse or other remote medical personnel, via postal mail, or via a telephone call from a nurse or other medical personnel.

The notification device or other response device detects a patient action in response to receiving the notification (80). Patient 12, for example, performs a specified action to indicate to a clinician or other programming operator receipt of the notification. For example, patient 12 interacts with an input medium of a response device in order to generate a response to the notification and send the response to the programming operator.

Alternatively, patient 12 performs a physical action that is detected by one or more sensors of the response device. In the example in which IMD 14 acts as the response device, patient 12 may bend over and return to an upright position in response to receiving the notification. One or more motion sensors of medical device 14 detect the bending and standing motion. In another embodiment, the action of patient 12 may be nothing, i.e., to sit or stand still for a period of time. Additionally, the patient action can be a manual action, such as visiting the clinic, mailing a response via postal mail, telephoning the clinic or the like.

In response to detecting the patient action, medical device 14 generates and sends a response to the notification to the programming operator (82, 84). Upon receiving the response from patient 12, the programming operator initiates the telemetry session with IMD 14. Specifically, in some embodiments, the programming operator interacts with the programmer to identify a set of operating parameters and sends instructions for updating operating parameters of the medical device (86). IMD 14 performs the instructions to update the operating parameters of the IMD (88).

Various embodiments of the invention have been described. These and other embodiments are within the scope of the following claims.

1. A method comprising:
notifying a patient that has a programmable medical device of initiation of a telemetry session with the programmable medical device; and
initiating the telemetry session in response to a patient action following the notification of the patient.

2. The method of claim 1, wherein the telemetry session comprises an interrogation session to extract data from the medical device.

3. The method of claim 1, wherein the telemetry session comprises a programming session to update one or more operating parameters of the medical device.

4. The method of claim 1, wherein notifying the patient comprises notifying the patient of initiation of a remote telemetry session with the programmable medical device.

5. The method of claim 1, further comprising:
generating an electronic notification to inform the patient of initiation of the telemetry session;
sending the electronic notification to a notification device; and
conveying the notification to the patient via the notification device.

6. The method of claim 5, wherein the notification device comprises one of the programmable medical device, a mobile phone, a pager, a personal digital assistant, a remote patient programmer, a remote patient monitor, a handheld programmer, a laptop computer, a desktop computer, a workstation, a telephone, and a web camera.

7. The method of claim 1, wherein notifying the patient comprises one of electrically stimulating tissue adjacent the programmable medical device, sounding an audible notification, displaying a visual notification, and emitting a fragrance.

8. The method of claim 1, wherein notifying the patient comprises one or notifying the patient via postal mail, and notifying the patient in person.

9. The method of claim 1, further comprising:
detecting performance of the patient action; and
sending a response to the notification upon detecting the patient action.

10. The method of claim 9, wherein detecting performance of the patient action comprises receiving input from the patient via an input medium of a response device.

11. The method of claim 9, wherein the response device comprises one of a mobile phone, a personal digital assistant, a remote patient programmer, a remote patient monitor, a handheld programmer, a laptop computer, a desktop computer, a workstation, a telephone, the programmable medical device, and a web camera.

12. The method of claim 9, wherein detecting performance of the patient action comprises sensing one or more parameters to detect a physical action performed by the patient.

13. The method of claim 12, wherein the physical action comprises one of holding breath for a specified period of time, breathing in a specified pattern for a specified period of time, tapping the medical device, tapping skin covering an implantable medical device, changing position of a body of the patient, shining a light on skin covering the medical device, swiping a magnet over the medical device, holding an antenna over the medical device, and turning on remote programmer.

14. The method of claim 1, wherein the programmable medical device comprises an implantable programmable medical device.

15. The method of claim 14, wherein the implantable programmable medical device comprises one of a pacemaker, an implantable cardioverter/defibrillator (ICD), a pacemaker/cardioverter/defibrillator (PCD), an implantable cardiac hemodynamic monitor, a subcutaneous, non-intravascular monitor, a neurostimulation device, and a drug delivery device.

16. The method of claim 1, wherein the programmable medical device comprises an external programmable medical device.

17. A system comprising:
a programmable medical device that performs at least one of diagnosing a patient, monitoring the patient, and delivering therapy to the patient; and
a notification device that notifies the patient of initiation of a telemetry session with the programmable medical device.

18. The system of claim 17, wherein the telemetry session comprises an interrogation session to extract data from the medical device.

19. The system of claim 17, wherein the telemetry session comprises a programming session to update one or more operating parameters of the medical device.

20. The system of claim 19, further comprising a programmer that initiates the programming session with the programmable medical device in response to a patient action.

21. The system of claim 20, further comprising a remote telemetry station that communicates with the clinician programmer to initiate a remote programming session in response to the patient action.

22. The system of claim 17, further comprising a communication device that generates an electronic notification to inform the patient of initiation of the telemetry session and sends the notification to the notification device.

23. The system of claim 17, wherein the patient interacts with the notification device to respond to the notification.

24. The system of claim 17, wherein the notification device notifies the patient of initiation of the telemetry session via one of electrically stimulating tissue adjacent the programmable medical device, sounding an audible notification, displaying a visual notification, emitting a fragrance, and vibrating the notification device.

25. The system of claim 17, further comprising a response device with which the patient interacts to respond to the notification.

26. The system of claim 25, wherein the response device detects performance of a patient action and sends a response to the notification device upon detecting the patient action.

27. The system of claim 26, wherein the response device further comprises an input medium via which the patient interacts with the response device, wherein the patient interacts with the input medium in response to receiving the notification.

28. The system of claim 27, wherein the input medium comprises one of a keyboard, a keypad, a stylus, a mouse, a button, a touch screen, a card reader, a biometric reader, and a voice recognition device.

29. The system of claim 26, wherein the response device comprises at least one sensor that detects a physical action performed by the patient.

30. The system of claim 29, wherein the sensor comprises one of a blood oxygenation sensor, an accelerometer, and an optical sensor.

31. The system of claim 29, wherein the physical action comprises one of holding breath for a specified period of time, breathing in a specified pattern for a specified period of time, tapping the medical device, tapping skin covering an implantable medical device, changing position of a body of the patient, shining a light on skin covering the medical device, swiping a magnet over the medical device, holding an antenna over the medical device, and turning on remote programmer.

32. The system of claim 25, wherein the response device comprises one of the programmable medical device, a mobile phone, a personal digital assistant, a remote patient programmer, a handheld programmer, a laptop computer, a desktop computer, a workstation, a telephone, and a web camera.

33. The system of claim 17, wherein the programmable medical device comprises an implantable programmable medical device.

34. The system of claim 33, wherein the implantable programmable medical device comprises one of a pacemaker, an implantable cardioverter/defibrillator (ICD), a pacemaker/cardioverter/defibrillator (PCD), an implantable cardiac hemodynamic monitor, a subcutaneous, non-intravascular monitor, a neurostimulation device, and a drug delivery device.

35. The system of claim 17, wherein the programmable medical device comprises an external programmable medical device.

36. The system of claim 17, wherein the notification device comprises one of the programmable medical device, a mobile phone, a pager, a personal digital assistant, a remote patient programmer, a handheld programmer, a laptop computer, a desktop computer, a workstation, a telephone, and a web camera.

37. A system comprising:
means for notifying a patient that has a programmable medical device of initiation of a telemetry session with the programmable medical device; and
means for initiating the telemetry session in response to a patient action following the notification of the patient.

38. The system of claim 37, wherein the telemetry session comprises an interrogation session to extract data from the medical device.

39. The system of claim 37, wherein the telemetry session comprises a programming session to update one or more operating parameters of the medical device.

40. The system of claim 37, wherein the programmable medical device comprises one of an implantable programmable medical device and an external programmable medical device.

41. A notification device comprising:
a communication unit that receives a notification of initiation of a telemetry session with a programmable medical device of a patient; and
an output medium to convey the notification to the patient to inform the patient of initiation of the telemetry session with the programmable medical device.

42. The notification device of claim 41, wherein the telemetry session comprises an interrogation session to extract data from the medical device.

43. The notification device of claim 41, wherein the telemetry session comprises a programming session to update one or more operating parameters of the medical device.

44. The notification device of claim 41, further comprising an input medium via which the patient interacts to respond to the notification.

45. The notification device of claim 44, wherein the input medium comprises one of a keyboard, a keypad, a mouse, a stylus, a button, a touch screen, a card reader, a biometric reader, and a voice recognition device.

46. The notification device of claim 41, further comprising at least one sensor to detect a physical action performed by the patient in response to the notification.

47. The notification device of claim 41, wherein the output medium forms part of one of the programmable medical device, a remote patient programmer, a mobile phone, a pager, a personal digital assistant, a laptop computer, a desktop computer, a workstation, a telephone, and a web camera.

48. A method comprising:
receiving an electronic notification of initiation of a telemetry session with a medical device of a patient;
conveying the electronic notification to the patient to inform the patient of initiation of the telemetry session with the programmable medical device; and
sending a response to the electronic notification upon receiving input from the patient.

49. The method of claim 48, wherein the telemetry session comprises an interrogation session to extract data from the medical device.

50. The method of claim 48, wherein the telemetry session comprises a programming session to update one or more operating parameters of the medical device.

51. The method of claim 48, wherein receiving input from the patient comprises detecting performance of a patient action.

52. The method of claim 48, wherein the patient action comprises one of holding breath for a specified period of time, breathing in a specified pattern for a specified period of time, tapping the medical device, tapping skin covering an implantable medical device, changing position of a body of the patient, shining a light on skin covering the medical device, swiping a magnet over the medical device, holding an antenna over the medical device, and turning on a remote programmer.

53. A method comprising sending an electronic notification to a patient that has a programmable medical device to notify the patient of initiation of a remote telemetry session with the programmable medical device.

54. The method of claim 53, wherein the telemetry session comprises an interrogation session to extract data from the medical device.

55. The method of claim 53, wherein the telemetry session comprises a programming session to update one or more operating parameters of the medical device.

56. The method of claim 53, further comprising:
receiving a response from the patient indicating receipt of the electronic notification; and
initiating the telemetry session upon receiving the response.

57. The method of claim 53, wherein the programmable medical device comprises one of an implantable programmable medical device and an external programmable medical device.

58. A programming device comprising:
a notification generator that generates an electronic notification; and
a communication unit that sends the electronic notification to a patient that has a programmable medical device to notify the patient of initiation of a programming session with the programmable medical device.

59. The programming device of claim 58, wherein the programming device receives a response to the electronic notification from the patient and initiates a programming session with the programmable medical device upon receiving the response from the patient.

60. The programming device of claim 59, wherein the programming session comprises a remote programming session.

61. The programming device of claim 58, further comprising an input medium via which a programming operator interacts with the programming device to generate the electronic notification.
